(19) United States
(12) Patent Application Publication
(10) Pub. No.: US 2022/0192549 A1
(43) Pub. Date: Jun. 23, 2022

(54) ORGANIC MULTI-CHANNEL OPTOELECTRONIC SENSORS FOR WEARABLE HEALTH MONITORING

(71) Applicant: The Regents of the University of California, Oakland, CA (US)

(72) Inventors: Yasser T. Khan, Berkeley, CA (US); Donggeon Han, Berkeley, CA (US); Jonathan KangYu Ting, Berkeley, CA (US); Maruf Ahmed, Berkeley, CA (US); Ana Claudia Arias, Berkeley, CA (US)

(21) Appl. No.: 17/691,437

(22) Filed: Mar. 10, 2022

Related U.S. Application Data
(63) Continuation of application No. PCT/US2020/053478, filed on Sep. 30, 2020.
(60) Provisional application No. 62/908,219, filed on Sep. 30, 2019.

(51) Int. Cl. A61B 5/1455; H01L 27/28; H01L 51/00

(52) U.S. Cl. CPC A61B 5/14551; H01L 27/288; H01L 51/0097; A61B 5/681; A61B 2562/164; A61B 2562/185; H01L 2251/5338

(57) ABSTRACT

A pulse oximeter device is provided that includes a first light emitting element configured to emit red light, a second light emitting element configured to emit green light or near infrared (NIR) light, and a sensor element configured to detect red and green light or detect red and NIR light. The sensor element may have a substantially circular geometry, and the first light emitting element and the second light emitting element may each have a substantially arc-shaped geometry, and wherein the sensor element is positioned between the first light emitting element and the second light emitting element, or the first and second light emitting elements together may have a substantially circular geometry, and wherein the sensor element comprises one or more substantially arc-shaped portions, and wherein the first and second light emitting elements are partially surrounded, or fully surrounded, by the sensor element.

### CROSS REFERENCES TO RELATED APPLICATIONS

This patent application is a continuation of PCT Application No. PCT/US2020/053478 by Yasser T. Khan et al., entitled “ORGANIC MULTI-CHANNEL OPTOELECTRONIC SENSORS FOR WEARABLE HEALTH MONITORING,” filed Sep. 30, 2020, which claims priority to U.S. Provisional Patent Application No. 62/908,219 by Yasser T. Khan, entitled “ORGANIC MULTI-CHANNEL OPTOELECTRONIC SENSORS FOR WEARABLE HEALTH MONITORING,” filed Sep. 30, 2019, each of which is incorporated herein by reference in its entirety.

### BACKGROUND

In the human body, cardiac rhythm changes the blood volume passing through the arteries, which generates a pulsatile signal that can be optically measured using a light source and a detector; this optical sensing technique is known as photoplethysmography (PPG). Generally, the PPG signal is used for calculating heart rate by utilizing only one light source, and for measuring oxygen saturation (SₚO₂) by employing two light sources. Pulse oximeters measure SₚO₂ of blood by using PPG signals at two distinct wavelengths where light absorption in oxygenated and deoxygenated blood is different. PPG and oximetry can be performed in both transmission and reflection mode. Conventionally, transmission-mode pulse oximeter sensors composed of solid-state light-emitting diodes (LEDs) and photodiodes (PDs) are used to measure SₚO₂ at the extremities of the body where light can easily penetrate thin regions of tissue, such as the earlobes and the fingertips. However, this method of measuring SₚO₂ presents a few limitations—(i) Transmission-mode oximetry has limited sensing locations, and (ii) Solid-state LEDs and PDs do not conform well to the skin, therefore, reduce the signal-to-noise ratio (SNR).

Over the past few years, flexible and wearable sensors are getting significant attention in both academic research and industry due to their skin conformable form factors. Consequently, flexible optical sensors have been extensively studied for PPG and oximetry as they enhance SNR and provide design versatility. Sensor fabrication and sensing methodology remain a strong focus of recent reports. However, the reflectance oximeter sensor design, which is a crucial component of reflection-mode PPG and oximetry, is not well-reported in the literature. In addition, wearable reflection-mode PPG sensors and oximeters are prone to different types of noise, such as motion artifacts (MAs), thermal noise, and electromagnetic interference. Thermal noise and electromagnetic interference are each a high-frequency noise and can be eliminated through filtering. MA, however, is challenging to remove from the PPG signals. Adaptive filtering and comparing PPG signal to a reference accelerometer signal are popular techniques for reducing MAs. Furthermore, multi-channel PPG signals can also be utilized to extract heart rate and oxygenation information from channels that are less affected by MAs. The multi-channel PPG approach does not require additional hardware blocks or a reference signal.

### SUMMARY

In various embodiments herein, varied reflectance oximeter sensor designs with varying device geometry, light emitter and detector spacing are provided. In certain embodiments, an optical barrier between the emitter and the detector is provided to maximize sensor performance. Additionally, in certain embodiments, a printed, flexible, two-channel reflectance oximeter is provided, which is able to collect PPG signals using red and near-infrared (NIR) (or green) organic light-emitting diodes (OLEDs) and organic photodiodes (OPDs). Inverse-variance weighting and template matching algorithms are used, in certain aspects, to improve the detection of heart rate from the multi-channel PPG signals. The various embodiments are useful for and in wearable smart watches and wristbands.

According to an embodiment, a pulse oximeter device is provided that includes a first light emitting element configured to emit red light, a second light emitting element configured to emit green light or near infrared (NIR) light, and a sensor element configured to detect red and green light or detect red and NIR light. The sensor element, in an embodiment, has a substantially circular geometry, and wherein the first light emitting element and the second light emitting element each have a substantially arc-shaped geometry, and wherein the sensor element is positioned between the first light emitting element and the second light emitting element. In another embodiment, the first and second light emitting elements together have a substantially circular geometry, and wherein the sensor element comprises one or more substantially arc-shaped portions, and wherein the first and second light emitting elements are partially surrounded, or fully surrounded, by the sensor element. In another embodiment, the sensor element has a substantially rectangular geometry, and wherein the first light emitting element and the second light emitting element each have a substantially bracket-shaped geometry, and wherein the sensor element is positioned between the first light emitting element and the second light emitting element. In another embodiment, the first and second light emitting elements together have a substantially rectangular geometry, and wherein the sensor element comprises one or more substantially bracket-shaped portions, and wherein the first and second light emitting elements are partially surrounded, or fully surrounded, by the sensor element.

In certain aspects, the first light emitting element comprises a first light emitting diode (LED), wherein the second light emitting element comprises a second LED, and wherein the sensor element includes a photodetector.

In certain aspects, each of the first and second LEDs comprises an organic LED, and wherein the photodetector comprises an organic photodiode.

In certain aspects, the device further includes a flexible substrate, wherein the first light emitting element, the second light emitting element and the sensor element are formed on the flexible substrate. In certain aspects, the flexible substrate comprises polyethylene napthalate (PEN).

In certain aspects, the sensor element is configured to detect light transmitted through tissue containing blood. In certain aspects, the sensor element is configured to detect light reflected by tissue containing blood.

In certain aspects, the device further includes a signal processing element, such as one or more processors with associated memory, configured to receive and process the signals representing detected red and green light or detected red and NIR light output by the sensor element to produce signals that represent blood oxygenation content.

In certain aspects, the device further includes a light blocking feature positioned between the sensor and at least one of the first light emitting element or the second light emitting element.

In certain aspects, the device further includes a light blocking feature positioned between the sensor and both the first light emitting element and the second light emitting element. In certain aspects, the light blocking feature includes a black tape.

In certain aspects, a spacing between the sensor and each of the first light emitting element and the second light emitting element is between about 2 mm and about 6 mm.

In certain aspects, a wearable sensor device comprising a pulse oximeter device according to any of the described embodiments is provided.

In certain aspects, a method of making a pulse oximeter device according to any of the described embodiments is provided.

Some embodiments further include a non-transitory computer-readable storage medium storing program code including instructions that, when executed by a processor or processors, cause the one or more processors to perform one of the methods of processing the signals representing detected red and green light or detected red and NIR light output by the sensor element to produce signals that represent blood oxygenation content, as described herein. Non-exclusive examples of non-transitory computer-readable storage media include any medium that can store program code, such as a USB flash drive, a removable hard disk, a read-only memory (ROM), a random access memory (RAM), a magnetic disk, or an optical disc.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF DRAWINGS

FIG. 1 illustrates aspects of a multi-channel reflectance PPG sensor according to an embodiment; panel (a) shows a schematic illustration of a two-channel (or two-pixel) wrist-worn reflectance PPG sensor according to an embodiment; panel (b) shows a setup for the multi-channel PPG sensor with two circular sensors spaced 4 cm apart to collect data from the ulnar artery (Ch 1) and the radial artery (Ch 2), with the sensor pixels driven using an AFE, while multiplexers are used to switch between the pixels—both red and NIR PPG signals are collected and processed for extracting HR and pulse oxygenation data; panel (c) is a photograph of the multi-pixel reflectance PPG sensor bent to a radius of curvature of 5 cm.

FIG. 2 illustrates a reflectance PPG sensor design and placement on the wrist, according to an embodiment: panels (a-c) show different sensor geometries with the same active areas; panel (a) shows a rectangular geometry (R), where the OLEDs are placed at either side of the OPD; panel (b) shows a bracket geometry (B), where the OLEDs are shaped as brackets and placed around the square OPD; panel (c) shows a circular geometry (C), where the OLEDs are shaped as block arcs and placed around the circular OPD; panel (d) shows a photograph of the printed reflectance oximeter sensor placed on the underside of the wrist, with the radial and ulnar artery sensing locations marked to show sensor placement locations; the inset shows a circular sensor with red and NIR OLEDs on the top and the bottom side of the OPD, respectively; panel (e) shows normalized electroluminescence (EL) of the red (red line) and NIR (peach line) OLEDs and EQE of the OPD (brick line)—the OPD shows similar EQE at both red and NIR wavelengths.

FIG. 3 shows PPG signal variation on the wrist: panel (a) shows three sensor placement locations—(i) on top of the wrist, (ii) on top of the ulnar artery, and (iii) on top of the radial artery; panel (b) shows PPG signals from the wrist, ulnar and radial arteries, with red color for the red channels and peach color for the NIR channels—since the signal on the wrist is weak, a 10× gain setting is used to resolve the pulsatile PPG signal; panel (c) shows PPG signal magnitudes at the wrist, ulnar and radial arteries—the error bars represent data from 3 separate trials.

FIG. 4 shows assembly of the printed sensor according to an embodiment: panel (a) shows a schematic depicting the sensor assembly, with polyethylene naphthalate used as the base substrate and inkjet-printed silver traces used to connect the optoelectronic sensor to the control electronics—red and NIR OLEDs and the OPD are then connected to complete the sensor; panel (b) shows a photograph of the assembled sensor.

FIG. 5 shows pulsatile signal magnitudes for different sensor geometries, according to embodiments: panel (a) shows photographs of the rectangular sensors with emitter-detector spacing, d=2, 4, 6 mm—the devices are labeled as R2, R4, and R6; panels (b-c), similar to panel (a), show photographs of the bracket and circular sensors with emitter-detector spacing, d=2, 4, 6 mm—the devices are labeled as B2, B4, B6, C2, C4, and C6; panel (d) shows the pulsatile signal magnitudes for all rectangular, bracket and circular sensors, where red-colored bars represent data for the red channel, while the peach-colored bars represent data from NIR channel—the error bars represent data from 3 separate trials.

FIG. 6 shows performance comparison of different sensor geometries: panel (a) shows photographs of the rectangular, bracket and circular sensors with an emitter-detector spacing of 2 mm; panel (b) shows PPG signals from the red and NIR channels for the sensors shown in panel (a); panel (c) shows the pulsatile signal magnitudes for the smallest rectangular, bracket and circular sensors, where red-colored bars represent data for the red channel, while the peach-colored bars represent data from NIR channel—the error bars represent data from 3 separate trials; panel (d) shows the schematic of the sensor with an optical barrier placed between the emitters and the detector; panel (e) shows pulsatile signal magnitudes of the red and NIR channels without and with the optical barrier; the error bars represent data from 3 separate trials.

FIG. 7 shows a process flow of the Template matching (TM) algorithm in panel (a) and the Inverse-variance weighting (IVW) algorithm in panel (b).

FIG. 8 shows heart rate detection using TM and IVW algorithms from a simulated dataset, according to an embodiment: panel (a) shows a 8 min long pristine PPG signal (Ch 1, top panel) and a PPG signal with 10 dB SNR (Ch 2, middle panel) are used as the multi-channel PPG data—calculated HR from Ch 1 and Ch 2 using TM and IVW algorithms and the ground truth are shown in the bottom panel; panel (b) shows zoomed-in data from 60 to 80 s of a—Ch 1, Ch 2, weighted PPG signal using TM and IVW algorithms, and calculated HR.

FIG. 9 shows heart rate detection using TM and IVW algorithms from the printed multi-channel PPG sensor, according to an embodiment: panel (a) shows the printed sensor placed on the underside of the wrist; panel (b) shows Ch 1 and Ch 2 are PPG data collected from the ulnar and the radial arteries, respectively—PPG_{w,TM} and PPG_{w,IVW} are the weighed PPG signals generated by TM and IVW algorithms—HR_{Ch1} and HR_{Ch2} are calculated heart rate by using Ch 1 and Ch 2 PPG data—the bottom panel shows calculated HR by TM and IVW algorithms.

FIG. 10 shows the effect of SNR.

FIG. 11 illustrates alternate sensor geometry embodiments, wherein OLED elements are located internally to OPD elements: panel (a) shows a bracketed geometry; and panel b) shows a circular geometry.

### DETAILED DESCRIPTION

### Reflectance Oximeter Sensor Geometries and Operation

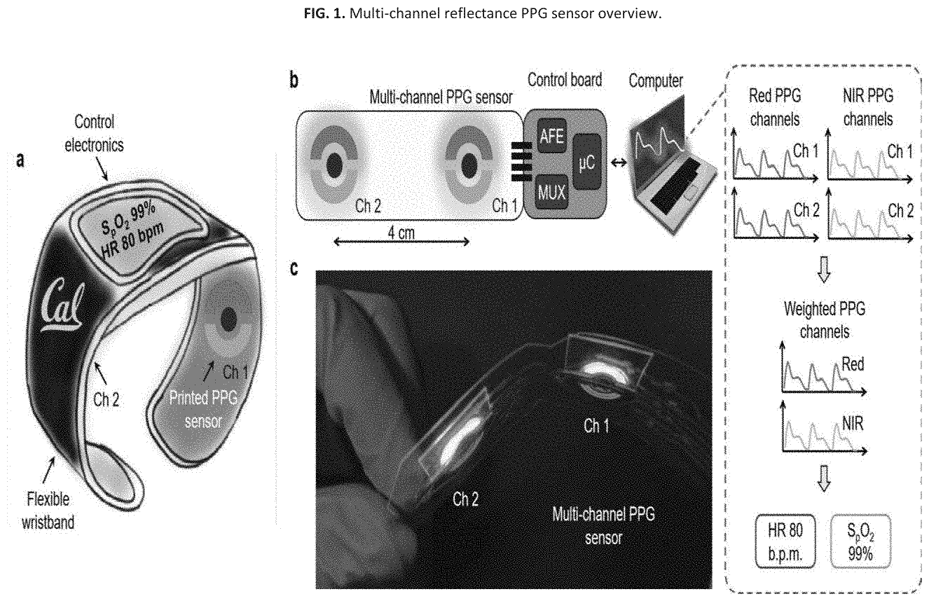

A schematic illustration of a two-channel (or two-pixel) wrist-worn reflectance PPG sensor, according to an embodiment, is shown in FIG. 1, panel (a). In this embodiment, the multi-channel sensor is designed using two circular sensors to collect PPG signals from the radial and the ulnar arteries as shown in FIG. 1, panel (b). The sensor is interfaced to multiplexers that switch between the pixels and connects to an analog front end (AFE). The AFE drives (e.g., sequentially drives) the OLEDs and reads out the OPD signals. Both red and NIR (or red and green) PPG signals are collected using the two pixels. As most wearable PPG sensors are wrist-worn, the two-channel PPG sensor is advantageous for use in on-wrist measurements. The underside of the wrist, especially on the radial and ulnar arteries, provide the best PPG signal magnitudes. One pixel (e.g., Ch 1) may be placed on the ulnar artery, while the other pixel (e.g., Ch 2) may be placed on the radial artery. A photograph of the multi-pixel sensor is shown in FIG. 1, panel (c), wherein the sensor is bent to a radius of curvature of ˜5 cm to resemble bending on the wrist.

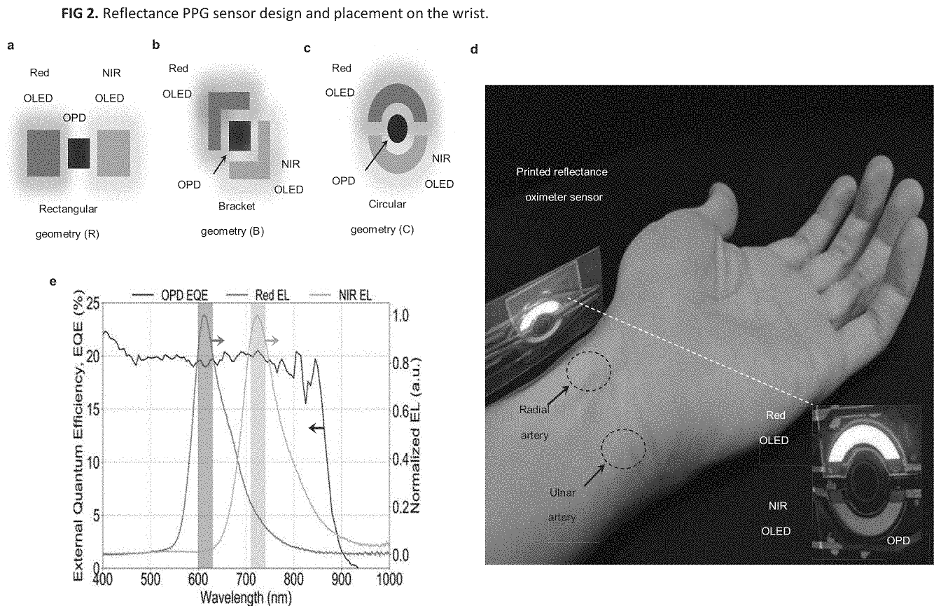

Reflection-mode sensors typically require light emitters and detectors assembled on a substrate or a circuit board. LEDs (e.g., red and NIR LEDs or red and green LEDs) are placed on either side of the PD to assemble the sensor. The designs of commercially available optoelectronic sensors are limited in shape—typically rectangular, which do not provide much versatility to vary the sensor geometry. On the other hand, printed optoelectronics can be fabricated in various shapes and sizes. Herein, three different sensor geometry embodiments are discussed as shown in FIG. 2, panels (a-c): (1) Rectangular geometry (R), where the OLEDs are placed at either side of the OPD; (2) Bracket geometry (B), where the OLEDs are shaped as brackets and placed around a square or rectangular OPD; (3) Circular geometry (C), where the OLEDs are shaped as block arcs and placed around a substantially circular OPD. The rectangular sensor design is similar to conventional sensors that use side-by-side optoelectronics placement. The bracket and the circular sensor geometries advantageously improve PPG SNR as will be shown in more detail below. It should be understood that the substantially circular sensor may include an elliptical or oval geometry. It should also be understood that a bracket-shaped element may include an L-shaped element with each branch having substantially the same length, e.g., as shown in FIG. 2, panel (b), or unequal branch lengths. Further, it should be appreciated that a bracket-shaped element may have sharp corners as shown in FIG. 2, panel (b), or it may have rounded corners, or a mix thereof. For example, the vertex may be rounded and the ends may be sharp, or the vertex may be sharp and the ends rounded. Similarly, the arc-shaped elements may have sharp corners as is shown in FIG. 2, panel (c), rounded corners. Moreover, the arc-shaped elements may be formed of discrete linear sub-segments which when combined resemble an arc-shaped geometry.

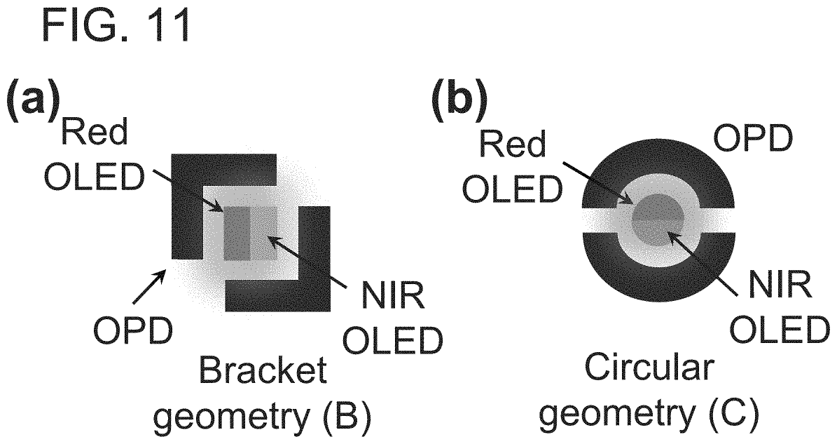

FIG. 11 illustrates alternate sensor geometry embodiments, wherein OLED elements are located internally to OPD elements. In FIG. 11, panel (a) shows a bracketed geometry, where one or more bracket-shaped OPDs are placed around, or partially around, e.g., at either side of, the two OLEDs, together forming a rectangular OLED pair; and panel b) shows a circular geometry, where the one or more OPDs are shaped as block arcs and placed around, or partially around, a substantially circular OLED pair. In certain embodiments, a sensor element, e.g., OPD, may fully surround an OLED pair.

FIG. 2, panel (d) shows an example sensor placement on the underside of the arm. Radial and ulnar arteries are marked to show sensor placement. In this example, the sensors are composed of printed red and NIR OLEDs with emission peaks at 630 and 725 nm respectively, and OPDs with external quantum efficiency (EQE) of ˜20% at the aforementioned wavelengths as shown in FIG. 2, panel (e). It will be appreciated that red and green OLEDs may be used and different OPDs may be used.

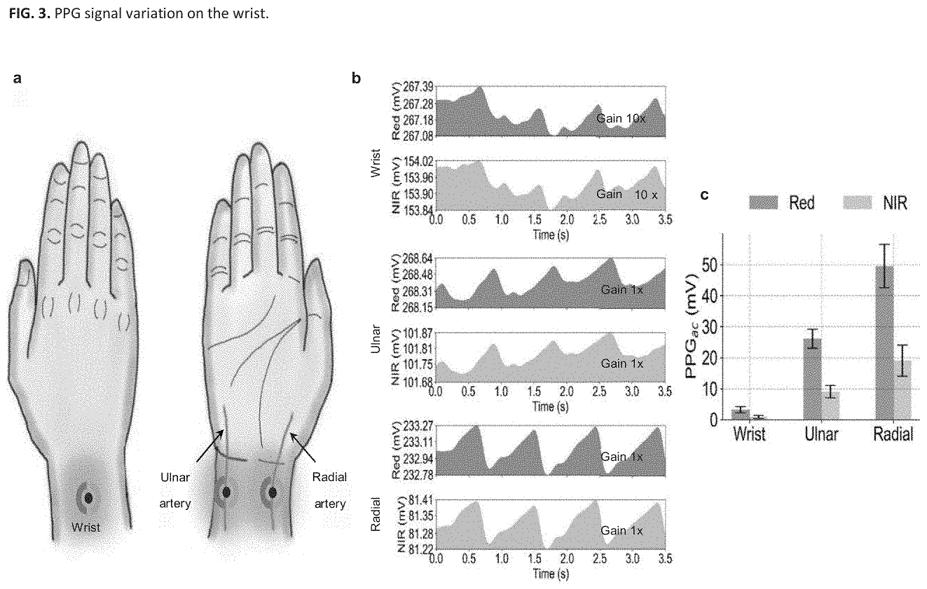

PPG signal magnitudes vary appreciably based on the sensor placement locations on the wrist. Accordingly, three sensing locations were explored: (i) On top of the wrist, (ii) on top of the ulnar artery, and (iii) on top of the radial artery as shown in FIG. 3, panel (a), and PPG signals recorded as shown in FIG. 3, panel (b). While the radial artery provided the cleanest signal (49.50 mV for red and 19.08 mV for NIR), the pulsatile PPG signal on top of the wrist was the weakest as shown in FIG. 3, panel (c). At the ulnar artery an order of magnitude improvement (26.12 mV for red and 9.02 mV for NIR) in PPG signal is observed over the wrist (3.24 mV for red and 0.94 mV for NIR). Therefore, a sensor on the underside of the wrist was used for both single and multi-channel measurements.

### Sensor Assembly and Calibration Examples

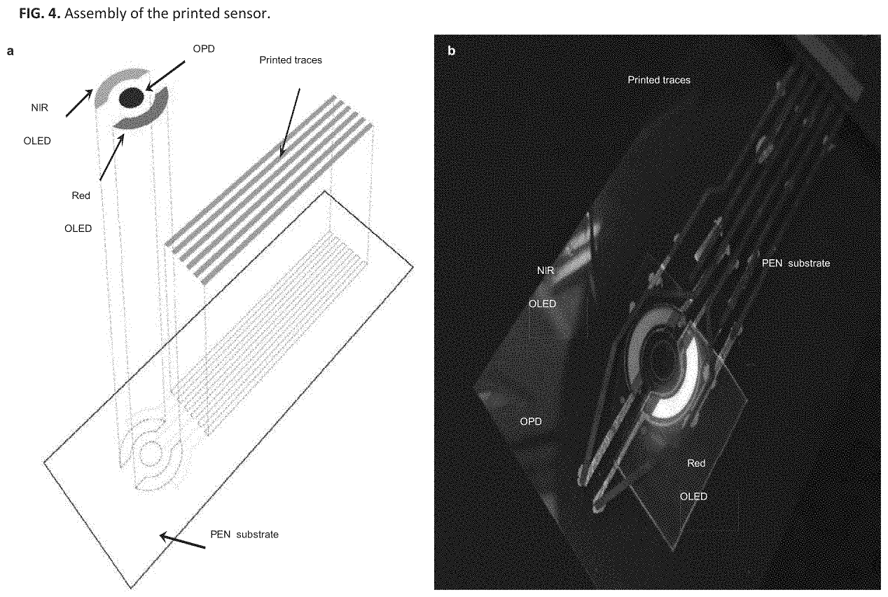

A base polyethylene naphthalate (PEN) substrate is used to assemble the reflectance sensor. Inkjet-printed silver traces are used to route connections from the optoelectronics to the control electronics, which include an AFE and a microcontroller with a universal serial bus (USB) interface to a computer. The OLEDs and the OPD are printed on separate plastic substrates and then assembled on the PEN substrate with silver traces as shown in FIG. 4, panel (a). The photograph of the assembled sensor with the OLEDs and the OPD is shown in FIG. 4, panel (b). Since different sensor geometries are compared, a two-step calibration is used to account for the batch-to-batch device variability of the OLEDs and the OPDs. A calibration platform composed of a silicon photodiode and a red LED is used to calibrate the assembled sensor. In the first step, the OLEDs are calibrated using the silicon photodiode by operating the OLEDs at a fixed current and recording the photodiode current. The OLEDs of the assembled sensor are turned on sequentially to measure their intensities using the silicon photodiode.

Each OLED is then calibrated to the maximum current measured in a batch of devices, K_{OLED}=max_{ISiPD}^{(ISiPD)}. In the second step, the OPDs are calibrated by recording the OPD current while running the solid-state red LED at a fixed drive current. The red LED of the calibration platform is turned on and the OLEDs are turned off for calibrating the OPDs. The fabricated OPD detects light from the red LED and the measured photocurrent is recorded. Similar to the OLEDs, each OPD is then calibrated to the maximum OPD current measured in a batch of devices, K_{OPD}=max_{IOPD}^{(IOPD)}. The obtained values are then used together with the measured PPG signal to calculate the calibrated signal magnitude. The calibration equation is given below, which is used to compare sensor performances for the three different geometries.

PPG_{cal}=KOLED•KOPD•PPGₘₑₐₛ[mV]

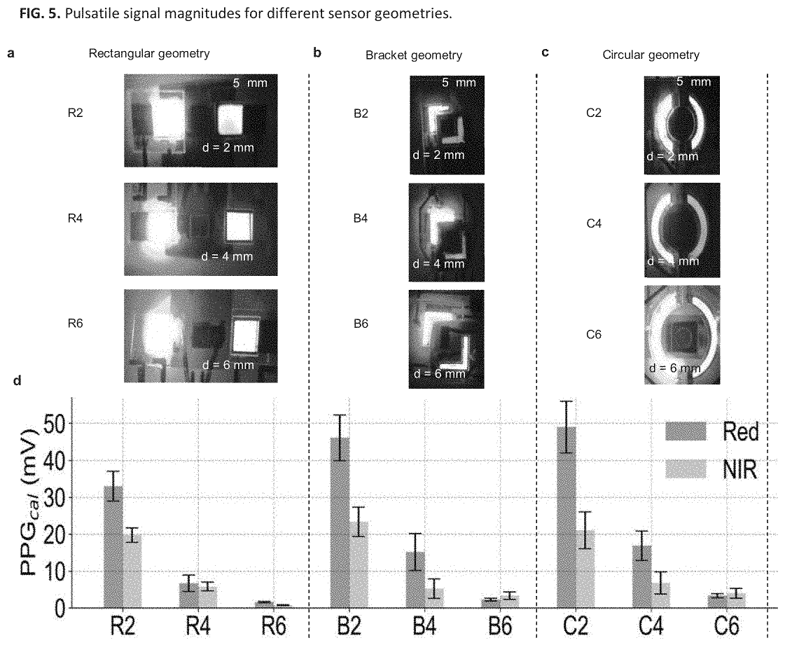

After the calibration step, a fair comparison among the three different geometries can be performed. Additionally, another important design parameter, emitter-detector spacing, d, is evaluated. FIG. 5, panels (a-c) show the photographs of the rectangular, bracket, and circular sensors, according to certain embodiments, with an emitter-detector spacing of 2, 4, and 6 mm, which are labeled as R2, R4, R6, B2, B4, B6, C2, C4, and C6. These labels are used in FIG. 5, panel (d) to show pulsatile PPG signal magnitude, PPG_{cal} based on Eq. 1.

The rectangular sensor includes OLEDs and an OPD that are all substantially square-shaped and which are placed side-by-side. Since the OLEDs do not surround the OPD from the top and the bottom, this scheme may be susceptible to ambient light, which could contribute to the noise of the measurement. Also, a significant amount of light coming out from the left edge of the red OLED and the right edge of the NIR OLED may not contribute to the measurement, and hence, gets lost. A perimeter light source that surrounds the OPD would be desired. The bracket design and the circular design schemes, where the light sources encompass the perimeter of the OPD, enhance measurement SNR. As shown in FIG. 5, panel (d), all three designs show an exponential decay with increasing d. Due to the perimeter lighting and better light collection by the OPD, both bracket and circular geometries outperform the rectangular design in terms of pulsatile PPG signal magnitude. When comparing the bracket and circular designs, a negligible difference in PPG_{cal} is observed.

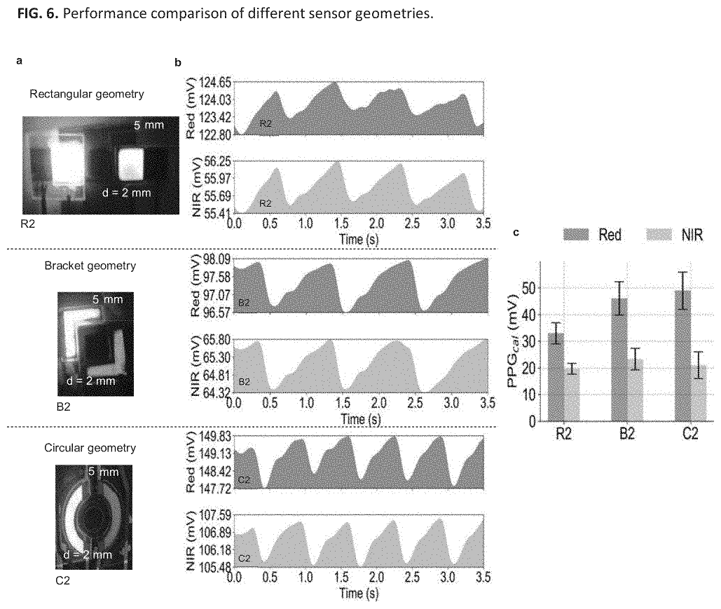

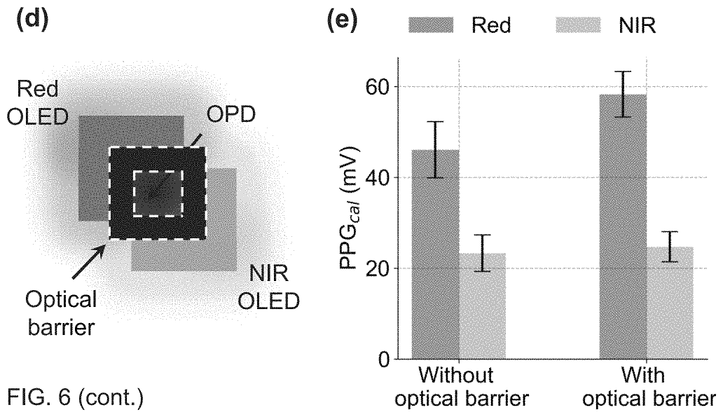

For a direct comparison of the different geometries, the emitter-detector spacing was kept constant at 2 mm, and the device area of the OLEDs and the OPDs was kept the same for all three geometries as shown in FIG. 6, panel (a). The OPD areas are kept fixed at 16 mm² and the OLED areas are kept fixed at 28 mm². The PPG waveforms for the different geometries are shown in FIG. 6, panel (b). Utilizing the bracket geometry, 39.7% and 18.2% improvements in PPG signal magnitude were observed in the red and NIR channels, respectively, over the rectangular geometry design. For the circular geometry design, 48.6% and 9.2% improvements in the red and NIR channels, respectively, were observed over the rectangular geometry design. The bracket and the circular designs exhibited similar performance in the PPG_{cal} as shown in FIG. 6, panel (c). Additionally, these two designs bring down the overall length of the sensor from 18.6 mm for the rectangular geometry to 12 mm for the bracket and 13.2 mm for the circular geometry.

In reflectance PPG and oximetry, the light coming back from the arteries contributes to the signal, while the light scattered from the skin surface contributes to noise. Therefore, blocking the light scattered from the skin surface enhances SNR. In an embodiment, a light blocking feature is incorporated in the design, e.g., by utilizing an optical barrier between the OLEDs and OPD. In an embodiment, black tape may be used, e.g., cut into the shape that fits the area between the OLEDs and the OPD and applied therebetween to block scattered light. FIG. 6, panel (d) shows the schematic of the sensor with an optical barrier placed between the emitters and the detector. With the blocking layer, a 26.5% improvement in PPG_{cal} was observed in the red channel, and PPG_{cal} remained almost the same in the NIR channel. Red light scatters more on the skin surface than the NIR. Therefore, more red light scattered by the skin surface gets blocked by the optical barrier, resulting in an improved red PPG_{cal} as shown in FIG. 6, panel (e), which shows pulsatile signal magnitudes of the red and NIR channels without and with the optical barrier; the error bars represent data from 3 separate trials.

### Multi-Channel PPG Sensor Operation and Data Processing

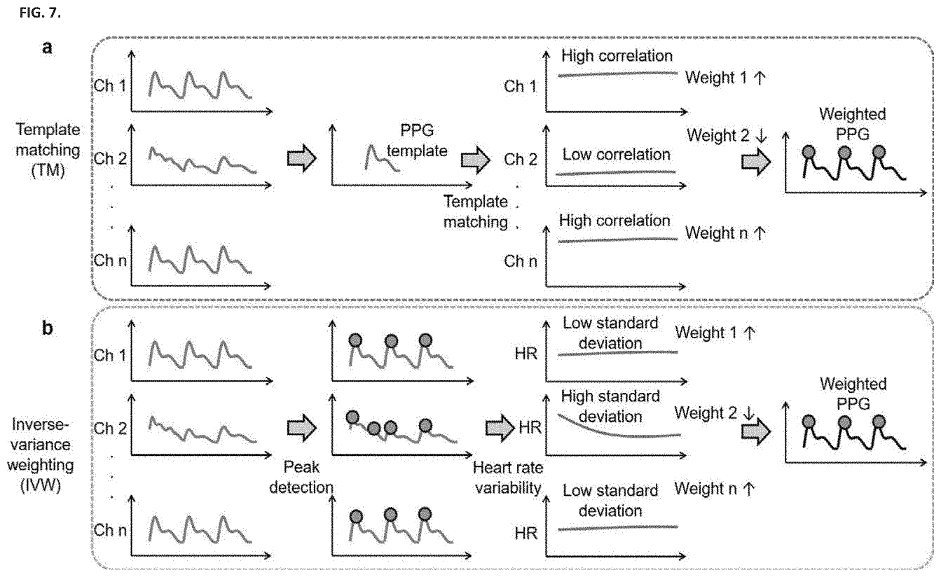

Wearable PPG sensors are susceptible to thermal noise, electromagnetic interference, and MAs. While thermal noise and electromagnetic interference can be reduced with filtering, reducing MAs requires additional hardware and software processing. Adaptive filtering is a popular technique for addressing MAs in PPG signals. Another approach is to simultaneously record PPG and a reference signal such as an accelerometer signal and apply hybrid algorithms to determine heart rate (HR) and pulse oxygenation. Multi-channel PPG acquisition and processing can also be used to reduce MA by utilizing channels that are lightly influenced by MA. Multiple PPG channels add redundancy to the measurement for signal quality assessment, which is important for properly extracting HR and pulse oxygenation values. To process multi-channel data, two algorithms may be used: (1) Template matching (TM) with an ideal PPG signal, and (2) Inverse-variance weighting (IVW). The efficacy of both methods in acquiring high-quality PPG signal, and extracting HR are examined. The process flow of the template matching and inverse-variance weighting algorithms are shown in FIG. 7, panel (a) and FIG. 7, panel (b), respectively.

Both TM and IVW algorithms are used to obtain a weighted PPG signal from multi-channel PPG. The equation for obtaining the weighted PPG is given in Eq. 2.

$${PPG}_{w} = \frac{{W}_{1} \times {PPG}_{1} + {W}_{2} \times {PPG}_{2} + … + {W}_{n} \times {PPG}_{n}}{{W}_{1} + {W}_{2} + … + {W}_{n}} \qquad ( 2 )$$

Here, PPG_{w} is the weighted PPG from all channels, Wᵢ is the weight for channel i determined by either of the two methods discussed in following subsections and PPGᵢ is the PPG signal from channel i.

Template Matching (TM) with an Ideal PPG Signal

Template matching is a popular data processing techniques in biomedical signal processing. TM has been widely used in processing electroencephalography (EEG), electrocardiography (ECG), and PPG data. An ideal PPG template was used to determine the fidelity of the signal from each channel. The ideal template can be obtained from experimental data or by modeling. A small window is acquired from each channel after filtering. Then, troughs are detected to find the pulses in each window. Next, the correlation coefficient of each pulse with the ideal template is calculated. If the correlation coefficient is positive, the correlation coefficient is used as a weight to calculate the weighted average of the two signals. If the correlation coefficient for a pulse is negative, the pulse is ignored, i.e., the weight for that pulse in that channel is set to zero. Thus, using this method, the weight W's in Eq. 2 can be given by Eq. 3.

$$\begin{matrix}{W}_{i} = {ρ}_{i} , if {ρ}_{i} \geq 0 , \\ = 0 , if {ρ}_{i} < 0\end{matrix} \qquad ( 3 )$$

Here, ρᵢ is the correlation coefficient between a pulse in channel i and the PPG template. HR and oxygenation values are determined from the weighted signal. The process flow for TM is presented in FIG. 7, panel (a).

### Inverse-Variance Weighting (IVW) Based on Heart Rate

In inverse-variance weighting algorithm, W, weight for channel i in Eq. 2 are assigned based on the standard deviation of heart rate variability in a specific time window. First, peaks and troughs of the signal from each window are determined. Then, the HR is calculated from the distances in between peaks or troughs. The channel with higher standard deviation in HR is assigned lower weight, because, in a small time-window of PPG signal, HR should not change too drastically. The weight assignment, in this case, is described by Eq. 4.

$${W}_{i} = \frac{1}{{σ}_{i}} \qquad ( 4 )$$

Here, σᵢ is the standard deviation of HR in channel i. After assigning the weight, the weighted average of the signals (PPG_{w}) is computed and the HR and other features are extracted from the signal. The process flow is presented in FIG. 7, panel (b).

### Implementation of TM and IVW Algorithms on a Simulated PPG Dataset

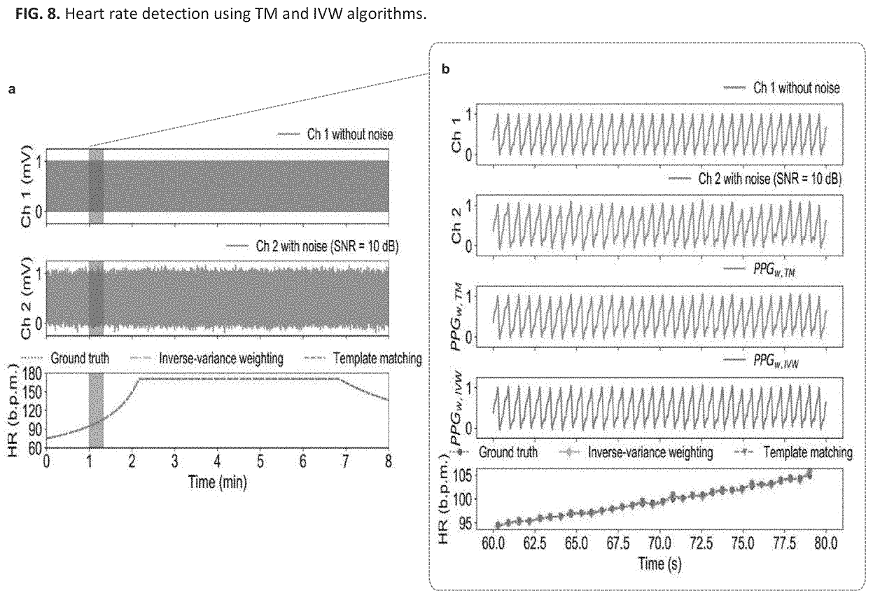

To test the efficacy of the TM and IVW algorithms, a simulated dataset is used to determine HR variability over time. The simulated dataset is designed to represent HR variability while performing an exercise as shown in FIG. 8, panel (a). Here, Ch 1 is a simulated PPG signal, where the HR goes up in the first few minutes, stays constant and then goes down slowly. This PPG signal represents the change in PPG during real-time exercise. Ch 2 is the same PPG signal with a low-frequency noise added to it. Slow and fast variations of HR are utilized. In the fast variation case, HR is varied from 71 beats per min (b.p.m.) to 200 b.p.m. in 20 s. Both TM and IVW algorithms successfully reconstructed the PPG signals and determine HR accurately. In the slow variation case, HR was varied from 95 to 105 b.p.m. in 20 s as shown in FIG. 8, panel (b), and both the algorithms successfully determined HR in this test case also.

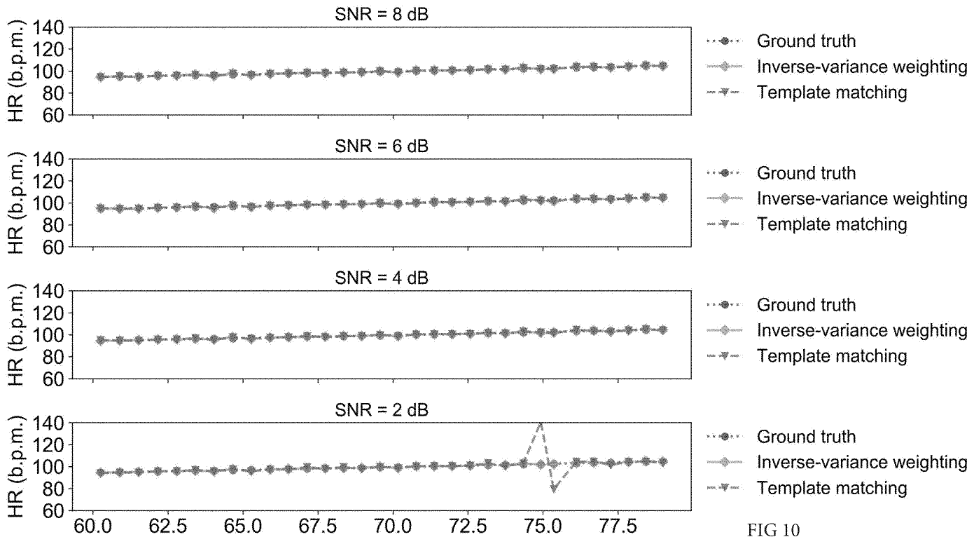

The effect of noise on the accuracy of determining HR using TM and IVW algorithms is examined by adding noise of frequency below 5 Hz to one of the channels. This frequency range of noise is chosen because noise with a frequency above 5 Hz can be removed from the signal using a low-pass filter (LPF). The effect of SNR is shown in FIG. 10. The SNR is varied and as long as the SNR is above 3 dB, both algorithms accurately detect HR. However, at SNR less than 3 dB, the TM algorithm fails to predict HR accurately.

In addition to the simulated dataset, three sets of PPG dual channel data reported by Zhang et al. (Zhang, Z., Pi, Z. & Liu, B. Troika: A general framework for heart rate monitoring using wrist-type photoplethysmographic signals during intensive physical exercise. IEEE Transactions on biomedical engineering 62, 522-531 (2015).) were used to test the efficacy of the TM and IVW algorithms. The results are summarized in Table 1, below. For the datasets, I and III the HR calculated using both methods are close to the ground truth HR, i.e., within about 2 b.p.m. However, in dataset II, both channels are more affected by MA, so the calculated HRs are further away from ground truth HR. For accurate detection of HR, at least one of the channels should be minimally affected by MA so that the PPG pulses are recognizable.

TABLE 1
Heart rate calculated from a literature dataset using template
matching and inverse-variance weighting algorithms.

| | Template | Inverse-Variance | Ground |
|---|---|---|---|
| Dataset | Matching | Weighting | Truth |
| I | 74.26 | 74.26 | 74.34 |
| II | 64.66 | 64.66 | 68.73 |
| III | 72.12 | 72.12 | 74.53 |

### Implementation of TM and IVW Algorithms on the Printed Multi-Channel PPG Sensor Data

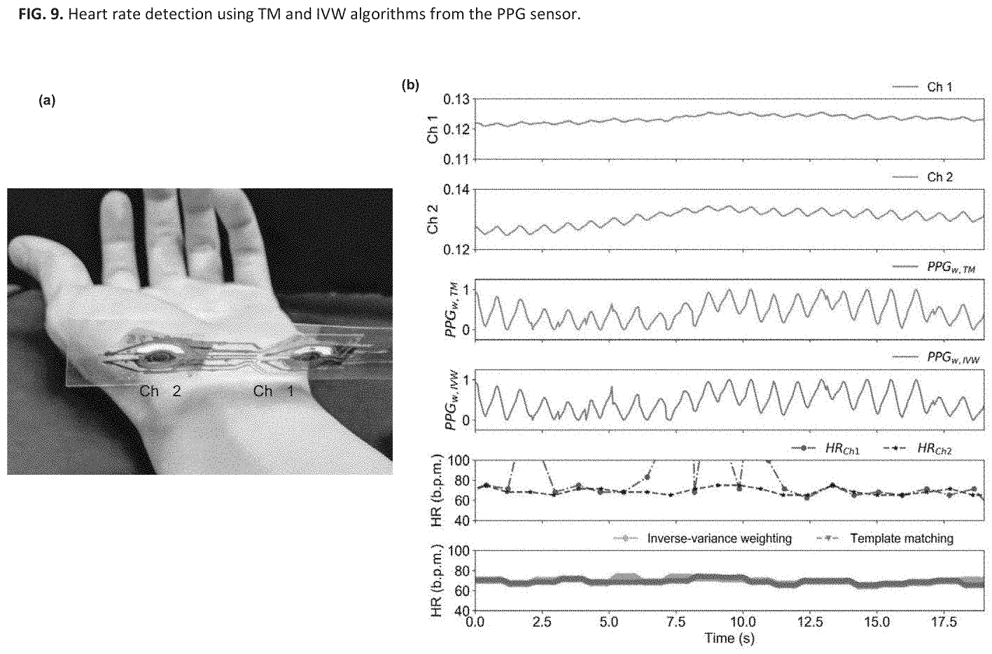

After validating the TM and IVW algorithms on the simulated and literature datasets, both methods were employed for processing the data collected by the printed multi-channel PPG sensor. The sensor is placed on the underside of the wrist, where Ch 1 collects data from the ulnar artery and Ch 2 collects data from the radial artery as shown in FIG. 9, panel (a) and FIG. 9, panel (b). The weighed PPG signals generated by TM and IVW algorithms are shown in panel 3 and 4 of FIG. 9b. Here, the signal magnitude of Ch 1 is weaker compared to Ch 2. Therefore, HR calculated using only using Ch 1 PPG signal demonstrates significant variation (panel 5 of FIG. 9, panel (b)). After implementing TM and IVW algorithms, accurate detection of HR is observed for both the algorithms (bottom panel of FIG. 9, panel (b)), demonstrating the feasibility of using these two methods for wearable PPG sensors and oximeters.

By utilizing the versatility of printed electronics, optoelectronic sensors for PPG and oximetry may advantageously be fabricated in different shapes and sizes. Embodiments herein provide novel and non-conventional geometries such as bracket and circular designs to improve sensor performance. These sensor geometries demonstrated a clear improvement over the conventional rectangular sensor design. These sensor geometries also not only improved the PPG signal magnitudes but also decreased the overall sensor length and reduced power consumption. These sensor designs coupled with multi-channel redundancy can be incorporated into wrist-worn devices, making them extremely promising for wearable reflectance PPG and oximetry.

### Example Methods and Components

### Fabrication and Characterization of the OLEDs

The OLEDs were printed on 125 μm thick indium tin oxide (ITO) patterned PEN substrates. The substrate was treated with (heptadecafluoro1,1,2,2-tetrahydrodecyl) (Gelest SIH5841.0) for 20 min under light vacuum (0.1-1 Torr) in order to make the surface hydrophobic. The substrate was then patterned by exposing the active area of the OLEDs with a plasma etcher to selectively etch off the hydrophobic layer. Then poly(3,4-ethylenedioxythiophene)-poly(styrenesulfonate) (PEDOT:PSS) (Clevios AI4083, Heraeus), interlayer, and the semiconducting polymers from Cambridge Display Technologies Ltd. (CDT) were subsequently blade-coated to form the emissive layer of the OLEDs. After all the layers were blade-coated, the samples were transferred to a thermal evaporator in a glovebox to evaporate calcium (99.5%, Strem Chemicals) and aluminum (99.999%, ACI Alloys) to finish the OLED stack. The shape of the top electrode determined the shape of the OLED emission. Each pixel was then encapsulated by face sealing using a UV curable epoxy (Delo Katiobond LP612) and plastic film (PQA1) on top. The details of the OLED fabrication process is described in details by Khan et al. (Khan, Y. et al. A flexible organic reflectance oximeter array. Proc. Natl. Acad. Sci. 115, E11015-E11024 (2018).).

### Fabrication and Characterization of the OPDs

The OPDs were printed on top of planarized PEN substrates (TeiJin PQA1) using blade coating techniques. The substrate was placed in a vacuum with 40 μL of heptadecaflouropolymer for 20 min to render substrate hydrophobic. A stainless steel stencil with cutouts of the desired PEDOT:PSS area was placed on top of the substrate, then treated for 1.2 min of oxygen plasma in a Diener Nano plasma system. Next, PEDOT:PSS was blade-coated, and the substrate was then annealed for 10 min at 120° C. 1:2 CDT Donor:PC70BM in 95:5 3,3′,5,5′-Tetramethylbenzidine:Benzyl Benzoate (TMB:BB) was blade-coated, and the substrate was then annealed for 1.5 h at 120° C. Finally, aluminum cathode at a base pressure of 5.10⁻⁶ Torr at a rate of 3-5 As⁻¹ was evaporated to finish the OPD stack. The details of the OPD fabrication process is described in details by Khan et al. (Khan, Y. et al. A flexible organic reflectance oximeter array. Proc. Natl. Acad. Sci. 115, E11015-E11024 (2018).).

### PPG Sensor Assembly and Calibration

A base PEN substrate was used to assemble the sensor. Inkjet-printed silver (ANP DGP 40LT-15C) traces were used to connect the OLEDs and the OPDs keeping an emitter-detector spacing of 2, 4, or 6 mm. Then fabricated red and NIR OLEDs and OPD pixels were connected to the traces using silver paste. The silver traces were connected to a flat flexible cable (FFC) connector for interfacing the sensor to the control electronics. To calibrate the PPG sensors, a calibration platform composed of a silicon photodiode (Hamamatsu 52387 series) and a red LED (5 mm clear red LED, 660 nm peak emission) were used. A two-step calibration was used to account for the batch-to-batch device variability of the OLEDs and the OPDs. In the first step, the OLEDs were calibrated using the silicon photodiode by operating the OLEDs at 10 mA cm⁻² and recording the photodiode current. Each OLED was then calibrated to the maximum current measured in a batch of devices. In the second step, the OPDs were calibrated by recording the OPD current while running the solid-state red LED at 0.7 mA drive current. Similar to the OLEDs, each OPD was then calibrated to the maximum OPD current measured in a batch of devices. The obtained values were then used together with the measured PPG signal to calculate the calibrated signal magnitude.

### Control Electronics for the Multi-Channel PPG Sensor

A Texas Instruments analog front end (AFE4490) was used to sequentially drive the OLEDs and read out the OPD signal. The two channels were selected using analog switches (Analog Devices ADG1608). The AFE was controlled with an Arduino Due microcontroller. The OLEDs were driven at 10 mA cm⁻² with a 9V battery in push-pull mode. A two-stage OPD gain circuitry was used to amplify the photocurrent. 100 kΩ feedback resistor was used in the first stage, and unity gain was used in the second stage. Finally, the data was collected using a Universal Serial Bus (USB) interface and was processed using a custom in-house software.

### Multi-Channel Data Collection and Processing

PPG data was collected from the two channels using the control electronics. Then, Template matching and inverse-variance weighting algorithms were used to obtain weighted PPG signals from multi-channel PPG. The weight assignment for the TM algorithm was calculated by Eq. 3, and the weight assignment for the IVW algorithm was calculated by Eq. 4. Then, the heart rate was calculated by timing the distances in between peaks or troughs of the weighted PPG signal.

U.S. Patent Application Publication No. 2017/0156651 A1, which is incorporated herein by reference, discloses various aspects of PPG and oximetry measurements, including reflectance-based measurements, as well as useful PPG device materials. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Various embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this specification includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A pulse oximeter device, comprising:
a first light emitting element configured to emit red light;
a second light emitting element configured to emit green light or near infrared (NIR) light; and
a sensor element configured to detect red and green light or detect red and NIR light,
wherein the sensor element has a substantially circular geometry, and wherein the first light emitting element and the second light emitting element each have a substantially arc-shaped geometry, and wherein the sensor element is positioned between the first light emitting element and the second light emitting element, or
wherein the first and second light emitting elements together have a substantially circular geometry, and wherein the sensor element comprises one or more substantially arc-shaped portions, and wherein the first and second light emitting elements are partially surrounded, or fully surrounded, by the sensor element.

2. The pulse oximeter device of claim 1, wherein a spacing between the sensor element and each of the first light emitting element and the second light emitting element is between about 2 mm and about 6 mm.

3. The pulse oximeter device of claim 1, wherein the first light emitting element comprises a first light emitting diode (LED), wherein the second light emitting element comprises a second LED, and wherein the sensor element includes a photodetector.

4. The pulse oximeter device of claim 3, wherein each of the first and second LEDs comprises an organic LED, and wherein the photodetector comprises an organic photodiode.

5. The pulse oximeter device of claim 1, further comprising a flexible substrate, wherein the first light emitting element, the second light emitting element and the sensor element are formed on the flexible substrate.

6. The pulse oximeter device of claim 5, wherein the flexible substrate comprises polyethylene napthalate (PEN).

7. The pulse oximeter device of claim 1, wherein the sensor element is configured to detect light transmitted through tissue containing blood.

8. The pulse oximeter device of claim 1, wherein the sensor element is configured to detect light reflected by tissue containing blood.

9. The pulse oximeter device of claim 1, further comprising a signal processing element configured to receive and process signals representing detected red and green light or detected red and NIR light output by the sensor element to produce signals that represent blood oxygenation content.

10. The pulse oximeter device of claim 1, further including a light blocking feature positioned between the sensor element and at least one of the first light emitting element or the second light emitting element.

11. The pulse oximeter device of claim 1, further including a light blocking feature positioned between the sensor element and both the first light emitting element and the second light emitting element.

12. The pulse oximeter device of claim 10, wherein the light blocking feature includes a black tape.

13. A pulse oximeter device, comprising:
a first light emitting element configured to emit red light;
a second light emitting element configured to emit green light or near infrared (NIR) light; and
a sensor element configured to detect red and green light or detect red and NIR light,
wherein the sensor element has a substantially rectangular geometry, and wherein the first light emitting element and the second light emitting element each have a substantially bracket-shaped geometry, and wherein the sensor element is positioned between the first light emitting element and the second light emitting element, or
wherein the first and second light emitting elements together have a substantially rectangular geometry, and wherein the sensor element comprises one or more substantially bracket-shaped portions, and wherein the first and second light emitting elements are partially surrounded, or fully surrounded, by the sensor element.

14. The pulse oximeter device of claim 13, wherein the first light emitting element comprises a first light emitting diode (LED), wherein the second light emitting element comprises a second LED, and wherein the sensor element includes a photodetector.

15. The pulse oximeter device of claim 14, wherein each of the first and second LEDs comprises an organic LED, and wherein the photodetector comprises an organic photodiode.

16. The pulse oximeter device of claim 13, further comprising a flexible substrate, wherein the first light emitting element, the second light emitting element and the sensor element are formed on the flexible substrate.

17. The pulse oximeter device of claim 13, wherein the sensor element is configured to detect light transmitted through tissue containing blood.

18. The pulse oximeter device of claim 13, wherein the sensor element is configured to detect light reflected by tissue containing blood.

19. The pulse oximeter device of claim 13, further comprising a signal processing element configured to receive and process signals representing detected red and green light or detected red and NIR light output by the sensor element to produce signals that represent blood oxygenation content.

20. The pulse oximeter device of claim 13, further including a light blocking feature positioned between the sensor element and at least one of the first light emitting element or the second light emitting element.
